(19) United States
(12) Patent Application Publication
(10) Pub. No.: US 2012/0165289 A1
(43) Pub. Date: Jun. 28, 2012

(54) TREATMENT

(75) Inventors: Jonathan Rhodes, Windsor (GB); Helen Simmons, Windsor (GB)

(73) Assignee: PROVEXIS (IBD) LIMITED, London (GB)

(21) Appl. No.: 13/129,644

(22) PCT Filed: Nov. 17, 2009
(86) PCT No.: PCT/GB2009/002688

§ 371 Date: Jan. 20, 2012

(30) Foreign Application Priority Data
Nov. 17, 2008 (GB) 0820972.8

(51) Int. Cl. A61K 31/736; A61P 31/04; A61P 1/12

(52) U.S. Cl. 514/54

(57) ABSTRACT

The invention provides composition comprising a therapeutically effective amount of a soluble fibre derivable from fruit of the Musa spp that may be used as a medicament for preventing or treating antibiotic associated diarrhoea (AAD). The soluble fibre is derivable from an aqueous solution decantable from homogenised fruit, and may be derived from boiling the fruit. The soluble fibre may be treated to remove starch. The compositions of the invention may be of use in treating AAD associated with C. difficile, and the therapeutically effective amount of soluble fibre may be an amount sufficient to inhibit adhesion of C. difficile. In addition to the compositions described above, the invention also provides a nutritional product for use in the prevention or treatment of antibiotic associated diarrhoea (AAD) comprising a therapeutically effective amount of a soluble fibre derivable from fruit of the Musa spp.

### TREATMENT

The present invention relates to the treatment of antibiotic associated diarrhoea (AAD) and in particular treatment of AAD with soluble extracts derived from edible plants.

AAD is a common complication of antibiotic therapy and can arise in up to 25% of subjects treated with antibiotics. AAD has been particularly associated with aminopencillin, cephalosporin and clindamycin therapy.

AAD is associated with abnormally loose bowel movements, abnormal fermentation processes in the gut; alterations in mucosal integrity; disruptions in mineral, fatty acid and vitamin metabolism and crampy abdominal pains. Some patients with AAD only present with relatively mild symptoms. However AAD can be very debilitating in some subjects and lead to electrolyte imbalances, dehydration, pseudomembranous coltis (PMC), toxic megacolon and even death.

AAD is believed to develop because the antibiotic treatment disrupts the equilibrium of the normal gut flora. This disruption allows pathogenic bacteria to flourish and thereby cause the abovementioned symptoms.

Clostridium difficile (C. difficile) infection has been increasing alarmingly, both in incidence and severity, in Westernised countries. The number of death certificates in England and Wales mentioning C. difficile infection have increased each year from 1999 to 2007. In 2007 there were 8,324 death certificates which mentioned C. difficile, a 28 percent increase from 2006 (Office for National Statistics, 2008). The risk of complicated C. difficile infection has, in part, been explained by the re-emergence of the BI/NAP1/027 strain of C. difficile. This strain has been demonstrated to elaborate high levels of toxin A and B. The majority of states in the US have had at least one hospital report of an outbreak due to this strain (Mc Farland L V. Nat Clin Pract Gastroenterol Hepatol 2008; 5:40-9).

C. difficile has been identified as one of the main pathogenic bacteria that may flourish in the gut after antibiotic treatment and C. difficile overgrowth is associated with the most serious adverse events associated with ADD (e.g. PMC). C. difficile caused diarrhoea is a significant health problem and can have particularly serious consequences in the old, immunocompromised, hospitalized adults and in children. Furthermore given the general increase in C. difficile infections (see above) diarrhoea associated with C. difficile overgrowth is becoming an increasing problem that needs addressing.

Current treatment options for subjects suffering from ADD include:
(a) the use of alternative antibiotics with a decreased risk of inciting diarrhoea;
(b) anti-diarrhoea drugs (e.g. loperaminde);
(c) administration of a probiotic (so-called “friendly” non-pathogenic bacteria;
(d) discontinuation of antibiotic therapy

Each of these known treatments are associated with specific disadvantages. For instance, the frequent swapping of antibiotics, (a) above, is associated with the development of antibiotic resistant bacterium and is therefore best avoided. The use of probiotics alone, (c) above, is not very effective when the diarrhoea is associated with inflammation and also have limited use when a patient already has diarrhoea and requires treatment. The discontinuation of antibiotic therapy (d) can be dangerous as the underlying infection may not be controlled.

It will be appreciated that none of the abovementioned treatment regimens successfully comprehensively address AAD and it is an object of the present invention to provide new and improved methods of preventing and treating AAD.

According to a first aspect of the present invention, there is provided a composition comprising a soluble fibre derivable from fruit of the Musa spp. for use as a medicament for the prevention or treatment of antibiotic associated diarrhoea (AAD).

According to a second aspect of the present invention, there is provided a method for the prevention or treatment of antibiotic associated diarrhoea (AAD) comprising administering to a subject in need of such prevention or treatment a therapeutically effective amount of a soluble fibre derivable from fruit of the Musa spp.

By “soluble fibre” we mean fibres capable of being dissolved in an aqueous medium (and also optionally the bloodstream) that comprise soluble polysaccharides or oligosaccharides from a non-starch source. Such fibres are capable of passing through the stomach and small intestine to the large intestine without being substantially digested. The fibres may then act as a fermentable substrate for bacteria in the large intestine. Different types of soluble fibre may be derived from common food sources such as fruits, especially apples and oranges; vegetables; oat bran; barley; and legumes. Soluble fibres from different sources will contain varying amounts of polysaccharides such as hemicelluloses or pectins, as well as varying amounts of oligosaccharides and monosaccharide derivatives. While the composition of soluble fibre is characteristic of the plant source, it will vary in response to factors such as cultivar, ripeness and geographical origin. Common food sources of insoluble fibre are cereal brans and fruits with edible skins and seeds, such as strawberries. Insoluble fibres aid digestion through various mechanisms including their ability to act as bulking agents (resulting in shorter transit time and increased faecal mass); and also hold on to water as they move through the intestinal tract (softening the stools and thereby helping prevent constipation).

By “antibiotic associated diarrhoea” (AAD) we mean diarrhoea caused in a subject by antibiotic treatment. ADD includes diarrhoea caused by abnormal growth of pathogenic bacterium following antibiotic treatment and in particular diarrhoea caused by abnormal growth of Clostridium difficile following antibiotic treatment.

It will be appreciated that compositions of the invention may comprise soluble fibre in pure form or alternatively it may also be mixed with other compounds (provided those compounds do not inhibit the efficacy of the fibre). Accordingly the present invention encompasses compositions comprising effective amounts of soluble fibre as-well-as insoluble fibre.

It is preferred that the soluble fibre used according to the invention is non-gelling.

Although we do not wish to be bound by any hypothesis the inventor believes that soluble fibres are useful in the treatment and prevention of AAD based upon their understanding of this scientific field and the work presented in Example 1.

The over growth of pathogenic bacteria, and particularly C. difficile, is associated with the fact that the normal gut flora is destroyed by the antibiotics and allows pathogens such as C. difficile to colonise the gut. The inventors have recognised that a critical step in bacterial colonisation of the gut is adherence of the bacterium to the mucosa through fimbrial- and/or surface proteins known as adhesins. These proteins act as lectins recognising glycosyl motifs expressed by host cell-surface glycolipid or glycoprotein and play a key role in bacterial pathogenicity. The inventors realised that an agent that prevents adhesion of pathogens such as C. difficile could be useful for reducing the toxic effects of the pathogen and thereby reduce the development of ADD after a subject has been treated with antibiotics.

The inventors therefore tested the ability of soluble fibres derived from fruits such as plantain for reducing adhesion of pathogens associated with AAD. Such fibres are known to control the invasion of the gut by E. Coli (see WO 2004/069143). However the inventors were surprised to find that the same soluble fibres, and particularly the derivatives discussed below were particularly effective for reducing the adhesion of C. difficile (see Example 1). This demonstrated that compositions according to the invention will inhibit C. difficile colonisation and will therefore prevent and/or treat AAD. Thus a preferred therapeutically effective amount of a soluble fibre to be used in accordance with the invention may be an amount sufficient to inhibit adhesion of C. difficile. A particularly preferred therapeutically effective amount of a soluble fibre may be an amount able to substantially prevent C. difficile adhesion.

Soluble fibre according to the present invention may be derived from a fruit, vegetable or cereal extract or an active fraction thereof. The active fraction may be derived from a fruit or grain selected from fruit or plants of the families Solanaceae, Rutaceae, Cucurbitaceae, Rosaceae, Musaceae, Anacardiaceae, Vitacease, Arecaceae, Ericaceae, Lauraceae and Poaceae.

Examples of fruits that can be used in accordance with the present invention are those selected from the families Solanaceae, Rutaceae, Cucurbitaceae, Rosaceae, Musaceae, Anacardiaceae, Bromeliaceae, Vitaceae, Arecaceae, Ericaceae and Lauraceae.

Examples of Solanaceae include the tomato, for example the English tomato variety. Examples of Rutaceae include the Citrus species such as Citrus paradisii (grapefruit), Citrus sinensis (orange), Citrus limon (lemon) and Citrus aurantifolia (lime). Examples of Cucurbitaceae include Cucumis melo (melon), e.g. the honeydew melon. Examples of Anacardiaceae include Mangifera indica (mango). Examples of Rosaceae include Pyrus sylvestris (apple), Pyrus communis (pear), Anygdalus perisca or Prunus persica Var. nectarina (nectarine), Prunus armeniaca (apricot), Prunus domestica (plum), Prunus avium (cherry), Prunus persica (peach), the strawberry and the blackberry. Examples of Musaceae include Musa paradisiaca (banana). Examples of Bromeliaceae include Ananas sativus (pineapple). Examples of Lauraceae include Persea gratisssima or Persea americana (avocado). Examples of Vitaceae include Vitis vinifera (grape). Examples of Arecaceae include Phoenix dactylifera (date). Examples of Ericaceae include the blueberry. Examples of Poaceae include Zea mays (maize), Sorghum vulgare (sorghum), Triticum aestivum (wheat) and Avena sativa (oats).

Particular examples of fruits, the extracts or active fractions of which have been found to be useful according to the invention are the banana, tomato, grapefruit, melon, mango, nectarine, strawberry, plum, grape, pear, apple and avocado. Particular examples of vegetables are the plantain, potato, carrot, parsnip, turnip, squash, courgettes and bell peppers. Particular examples of cereals are wheat grains, barley grains, maize kernels, oats and rice grains.

However the inventors have found that soluble fibres derived from the Musaceae family (i.e the Musa and Ensete genera) are particularly useful. Eleven Musa species are known but the soluble fibres are preferably derived from the cultivars Musa acuminata, Musa balbisiana, Musa paradisiaca or Musa sapientum.

A preferred soluble fibre for use in treating or preventing AAD is derived from plantain or green (i.e. unripe) edible bananas.

Soluble fibre according to the invention may be prepared in its simplest form by homogenising a source of the fibre (e.g. plantain flesh) in an aqueous solution and then decanting off the aqueous supernatant. This supernatant may then be used according to the invention. Alternatively the mixture may be centrifuged to separate solids from the aqueous solution comprising the soluble fibre. This aqueous solution may consist essentially of the juice of the fruit, optionally with the addition of extra water added during the homogenising step. Such aqueous extracts can be concentrated, enriched or condensed by, for example, standard techniques, e.g. evaporation under reduced pressure. Examples of concentrates are those which are at least 2-fold concentrated, more usually, at least 4-fold, for example at least 8-fold, or at least 40-fold, or at least 100-fold, or at least 200-fold, or at least 1000-fold.

The aqueous solution may be fractionated to isolate one or more active fractions comprising the soluble fibre therein by, for example, molecular weight filtration, or chromatography on a suitable solid support such as a sepharose gel (for size exclusion chromatography) or ion-exchange column using HPLC on a suitably treated silica or alumina, for example ODS coated silica; or by solvent extraction.

Soluble fibre according to the invention may be prepared by following one or more of the followings steps:

(A) The soluble fibre may be prepared by mashing or slicing fruit (e.g. plantain or banana) and then boiling the fruit in water (preferably sterile water) for between 2 and 60 minutes, preferably about 30 minutes. Alternatively dried fruit may be milled and then boiled in water as discussed above. After boiling, the solution should be centrifuged (e.g. at 10,000 g for 10 minutes) to separate insoluble material from the supernatant. The pellet should then be discarded and the supernatant may be used as soluble fibre according to the present invention. The fact that the supernatant is useful is particularly surprising in the light of the prior art which suggests that the pulp (i.e the pelleted material) may be medically useful. For instance, Rabbini et al. (2001) Gastroenterology 121 p 554-560) suggest banana pulp may be used to treat diarrhoea.

However, it should be appreciated that the inventor has found that boiling at 100° C. is not essential for preparing soluble fibre according to the invention.

(B) Preferred soluble fibre may undergo a treatment step with an enzyme capable of hydrolysing starch and thereby removing starch from the soluble fibre. Starch-degrading enzymes such as α-amylases from animal, bacterial or fungal sources, amyloglycosidases or pullulanases may be used for such a purpose, individually or in combination. Accordingly a preferred protocol for producing the soluble fibre involves the soluble fibre being extracted from boiled plantains and being treated with a starch digesting enzyme, or enzymes, to remove starch and produce a Non-Starch Polysaccharide (NSP) fraction.
(C) A further step that may be employed in the preparation and enrichment of soluble fibre according to the invention, is a precipitation step. Such a step may be employed to precipitate the soluble fibre in order that it may be separated from other water soluble contaminants. The precipitated soluble fibre may then be kept in powder form (see below) or alternatively may be resuspended in a liquid of a defined composition and volume (thereby regulating the concentration of the soluble fibre). The inventor has found that an ideal way of precipitating the soluble fibre is to add 80% ethanol to a crude extract of the soluble fibre. As an alternative to precipitation it will be appreciated the dialysis may be employed.

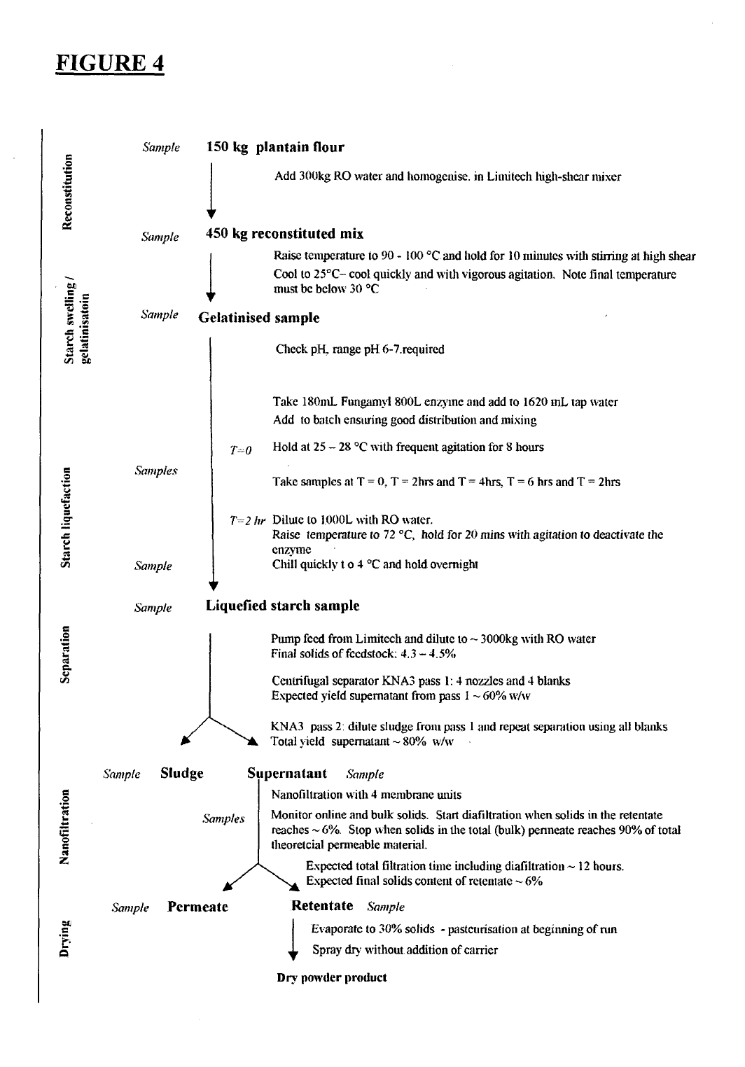

The abovementioned procedures are useful for relatively small-scale production of compositions according to the invention. However most preferred compositions may be formed using the techniques outlined below and in Example 2 that are also well suited to industrial scale-up. Accordingly a preferred method for making a free-flowing powder composition according to the present invention is outlined in Example 2 and in FIG. 4. This method represents an important feature of the invention. Therefore according to a further aspect of the invention there is provided a method of extracting non-starch soluble fibre from dry flour comprising the steps of:

(1) reconstituting the flour;

(2) Starch swelling and gelatinisation;

(3) Starch liquefaction;

(4) Separation of soluble and insoluble non-starch polysaccharides;

(5) Removal of low-molecular weight starch degradation products; and

(6) Drying.

### Stage 1: Reconstitution.

The dry flour (preferably from banana or plantain) should be weighed and mixed in a high shear mixer with reverse-osmosis purified water (RO water) in a ratio of about 1:2 until a homogenous suspension is formed.

### Stage 2: Starch Swelling and Gelatinisation

The homogenised mixture is heated to between 90 and 100° C. and held at temperature for about 10 minutes (preferably with continuous high-shear mixing). The starch present in the plantain flour swells and gelatinises with heating, and forms a very viscous elastic gel. Continuous high-shear mixing breaks the gel and prevents the mixture from sticking to the sides of the vessel during the heating step. The vessel should them be cooled (e.g. by means of cooling water) to about 25° C.

### Stage 3: Starch Liquefaction

An α-amylase (preferably a fungal α-amylase such as Fungamyl, Novozymes Corporation) is then used to liquefy the starch gel. A fungal source of α-amylase is preferred because it is a low-temperature enzyme with an optimum operating temperature of 25-28° C., at pH 6-7. High temperatures for extended periods result in polysaccharide degradation and are avoided; thus the low temperature enzyme is ideal. Liquefaction involves breaking α-1,6 bonds between glucose units in the starch molecules, to produce maltodextrins. α-1,4 bonds are not broken, as fungal α-amylase does not possess this catalytic activity. On addition of enzyme, the starch gel is immediately and irreversibly broken, as the starch molecules are degraded to smaller molecular weight maltodextrins. The liquefaction process is complete within about 2 hours at 25-28° C., the optimal temperature range for fungal α-amylase. After 2 hours, the liquefied mixture is diluted as required with RO water, and heated (e.g. to 72° C. for 20 minutes) to fully inactive the Fungamyl enzyme and to pasteurise the mixture. The mixture is then chilled quickly to 25-30° C.

### Stage 4: Separation of Soluble and Insoluble NSP

The mixture may be further diluted with RO water so that the final mixture contains 2.0-10.0% solids and more preferably 4.3-4.5% solids. The mixture is then separated on a centrifugal separator in two steps, so that an overall split of supernatant: sludge of 80%:20% (w/w) is achieved. The supernatant contains about 3.4-3.5% total solids, but <0.1% insoluble solids. The sludge contains 4.7-4.8% total solids, of which >50% are insoluble solids.

### Stage 5: Removal of Low-Molecular Weight Starch Degradation Products

A small amount of free glucose and maltose may be produced during the Fungamyl enzyme digestion. These, as well as other low molecular weight compounds such as sucrose, free amino acids, are removed from the soluble NSP by nanofiltration (e.g. using spiral-wound membranes). The membranes permit small molecules (MW<300 Da) to pass through, while retaining all other material inside the membrane. Thus water is removed from the mixture concomitantly, and unless replaced (a process called ‘diafiltration’), concentration of the mixture will occur.

Nanofiltration is carried out without diafiltration until the % solids of the retained material has risen from 3.4-3.5% to 6.0%. During this period, the theoretical amount of permeable material is calculated from the % solids content and flow rate of the permeate stream. Once the retentate has reached 6.0% solids, diafiltration is commenced, adding water back into the retentate at the same rate as it is being removed. This continues until 90% of the theoretically permeable material has been removed, at which point the process is halted. The retentate is recovered, and the permeate is discarded.

### Stage 6: Drying

The retentate fraction, containing soluble NSP and large MW maltodextrins, is concentrated (e.g. by evaporation under reduced pressure at a temperature of 40 C) until it reaches 30% solids. It is then passed to a spray-drier, incorporating an on-line pasteurisation step before it reaches the drier. The mixture is spray-dried without agglomeration to produce a fine dry powder with a particle size distribution of 50-100μ and a bulk density of 175 g/L.

The inventor has also found that an effective pH for a soluble fibre solution is around pH 7. An increase in pH increases solubility but excessive alkalinity was found to degrade the polysaccharides or oligosaccharides in the soluble fibre and result in a decrease in efficacy.

Table 1 illustrates the sugar content of a typical soluble fibre obtained according to the methods discussed above. Analysis of the sugar content of a soluble fibre provides a “fingerprint” for soluble fibre from a particular source. It is possible to distinguish between soluble fibre derived from plantain and other plants (e.g. pea-husk or wheat). It will therefore be appreciated that preferred Musa derived soluble fibre has a sugar content similar in range to that given in Table 1.

TABLE 1
Non-Starch Polysaccharide Constituent Sugars (g/100 g dry matter)

| Rhamnose | Fucose | Arabinose | Xylose | Mannose | Galactose | Glucose | Uronic acids |
|---|---|---|---|---|---|---|---|
| 2.1-3.2 | 2.1-3.6 | 8.8-11.5 | 5.9-7.2 | 17.6-21.0 | 8.7-10.3 | 22.5-27.8 | 22.5-26.7 |

It is more preferred that the sugar content is similar to that given in Table 2:

TABLE 2
Non-Starch Polysaccharide Constituent Sugars (g/100 g dry matter)

| Rhamnose | Fucose | Arabinose | Xylose | Mannose | Galactose | Glucose | Uronic acids |
|---|---|---|---|---|---|---|---|
| 2.6 | 2.8 | 8.6 | 6.4 | 18.9 | 9.2 | 25.4 | 25.1 |

Most preferred soluble fibre preparations for use according to the invention are described in Example 2. The soluble fibre preparation methods and the soluble fibres produced by such methods both represent preferred embodiments for use in accordance with the invention.

The compositions according to the invention are useful for preventing or treating any diarrhoea caused in a subject by antibiotic treatment and are particularly useful for preventing or treating diarrhoea caused by abnormal growth of C. difficile following antibiotic treatment. The compositions are useful for treating pre-existing diarrhoea and may be administered to a subject when diarrhoea commences, or may preferably be given within 1, 2, 3, 4, 5 or 6 days of diarrhoea commencing. In a preferred embodiment of the invention the composition is used as a prophylactic to prevent diarrhoea developing. For instance it may be given before onset of diarrhoea at any time during antibiotic therapy. Preferably the composition may be given at the time antibiotic therapy begins. Alternatively it may be given up to 12 hours before antibiotic therapy commences; up to 24 hours before antibiotic therapy commences or up to 2-5 days before antibiotic therapy commences.

AAD may be treated using soluble fibre derived from a natural source (e.g. prepared from Banana or plantain). Alternatively synthetic sugars may be used with the same, or similar composition to the soluble fibre prepared from natural sources.

It will be appreciated that a suitable crude preparation may be derived from boiling, or even just homogenising fruit in an aqueous solution. Fruit solids may be pelleted by centrifuging and the supernatant (containing soluble fibre) may be used according to the invention.

Clinical needs may dictate that such a crude preparation (e.g. the supernatant mentioned above) may need to be used substantially “neat” or even diluted. When this is the case the supernatant (whether diluted or not) may be mixed with a number of other agents that may be added for nutritional reasons, medical reasons or even for the purposes of adjusting the palatability of the soluble fibres for consumption by the subject being treated.

For instance, the soluble fibre may be formulated with a diary product (e.g. milk, a milk shake or yoghurt) or a fruit juice (e.g. orange juice or similar) to produce a palatable drink/beverage with the added benefit that it contains soluble fibre and therefore will be highly suitable as a refreshment for sufferers of AAD. Such products may be given to a subject in advance of antibiotic therapy in order that the soluble fibre may have a prophylactic effect.

Alternatively, the crude preparation may be included in a nutritional liquid for enteral feeding. For instance, the supernatant may be mixed with saline or an aqueous solution (other vitamins, minerals and nutrients may be included) for enteral feeding of subjects. It is preferred that such enteral feed may be given to a subject 48 hours, preferably 24 hours, or more preferably at least 12 hours in advance of antibiotic therapy in order that the soluble fibre may have a prophylactic effect.

It will be appreciated that concentration of the crude preparation from the first protocol may be required or alternatively a powder composition is desired. When this is the case the crude extract/supernatant will need to be concentrated/dehydrated.

Compositions comprising soluble fibre may be formulated as powders, granules or semisolids for incorporation into capsules. For presentation in the form of a semisolid, soluble fibre can be dissolved or suspended in a viscous liquid or semisolid vehicle such as a polyethylene glycol, or a liquid carrier such as a glycol, e.g. propylene glycol, or glycerol or a vegetable or fish oil, for example an oil selected from olive oil, sunflower oil, safflower oil, evening primrose oil, soya oil, cold liver oil, herring oil, etc. This may then be filled into capsules of either the hard gelatine or soft gelatine type or made from hard or soft gelatine equivalents, soft gelatine or gelating-equivalent capsules being preferred for viscous liquid or semisolid fillings.

Powders comprising soluble fibre according to the invention are particularly useful for making pharmaceutical or nutritional products that may be used to prevent or treat AAD.

Freeze-drying or spray drying represent preferred methods for producing a powder comprising soluble fibres according to the invention. Spray drying results in free-flowing granular powder mixes with good flow properties and quick dissolving characteristics.

It will be appreciated that spray-dried or freeze-dried powder produced by the protocols discussed above represent preferred powdered soluble fibre according to the invention. A preferred powder is derived from a reconstituted soluble fibre solution produced by steps (i)-(v) (see above) which is subsequently freeze-dried or spray-dried.

Powdered soluble fibre may be reconstituted as a clear/translucent low viscosity drink/beverage. Reconstitution may be into water or dairy or fruit juices as discussed above. It will be appreciated that the powder may be packaged in a sachet and reconstituted as a drink by a subject when required or desired.

Powder mixes represent preferred embodiments of the invention. Such mixes comprise powdered soluble fibre (as described above) mixed with further ingredients. Such ingredients may be added for nutritional or medical reasons or for improved palatability. The powdered soluble fibre may be mixed with granulated sugars of varying particle sizes to obtain free-flowing powder mixes of varying sweetness.

Alternatively natural sweeteners or artificial sweeteners (e.g. aspartame, saccharin and the like) may be mixed with the powdered soluble fibre for reconstitution as a low calorie/reduced calorie sweetened drink. The powder mix may comprise a mineral supplement. The mineral may be any one of calcium, magnesium, potassium, zinc, sodium, iron, and their various combinations.

Powder mixes may also contain buffering agents such as citrate and phosphate buffers, and effervescent agents formed from carbonates, e.g. bicarbonates such as sodium or ammonium bicarbonate, and a solid acid, for example citric acid or an acid citrate salt.

The soluble fibre can be presented as food supplements or food additives, or can be incorporated into foods, for example functional foods or nutraceuticals. Such products may be used as staple foods as well as under circumstances where there may be a clinical need.

The powders may be incorporated in to snack food bars for example fruit bars, nut bars and cereal bars. For presentation in the form of snack food bars, the powder can be admixed with any one or more ingredients selected from dried fruits such as sundried tomatoes, raisins and sultanas, ground nuts or cereals such as oats and wheat.

It will be appreciated that soluble fibre may advantageously be formulated as a pharmaceutical for use as a medicament (requiring a prescription or otherwise).

Powdered soluble fibre or concentrated liquid soluble fibre may also be incorporated into tablets, lozenges, sweets or other food-stuffs for oral ingestion. It will also be appreciated that such powdered soluble fibre or concentrated liquid soluble fibre may be incorporated into slow-release capsules or devices which may be ingested and are able to release soluble fibre into the intestines over a long period of time.

Soluble fibres may also be microencapsulated. For instance encapsulation may be by calcium-alginate gel capsule formation. Kappa-carrageenan, gellan gum, gelatin and starch may be used as excipients for micro-encapsulation.

Crude preparations, liquid concentrates, powders and the like may be combined with known therapeutic agents for treating AAD. As such the soluble fibre according to the invention may be used in a very effective combination therapy. It will be appreciated that the soluble fibre in solution may act as an ideal vehicle for other therapeutic agents (e.g. anti-diarrhoea drugs) for treating AAD

The soluble fibre may also be included in combination/synbiotic therapies that include a probiotic portion. The bacteria contained within many probiotic mixtures are thought to confer health benefits by boosting the populations of beneficial gut bacteria at the expense of non-beneficial bacteria, whereas the investigators hypothesise that soluble fibre confers benefits by preventing pathogen adhesion to the gut epithelium. Work carried out by the inventors has shown that species of probiotic bacteria (Bifidobacteria, Lactobaccilli and Streptococci) do not easily degrade soluble fibre according to the invention. Thus a mixture comprising a probiotic bacterial species and soluble plantain NSP in, for example, a yoghurt matrix has additive or even synergistic benefits, due to the different benefits conferred by its constituents. Therefore a preferred composition according to the invention comprises Bifidobacteria and/or Lactobaccilli and/or Streptococci and plantain soluble fibre. That said, compositions in accordance with the invention are able to treat AAD without the need for incorporation of bacteria, and such compositions without bacteria are an aspect of the invention having great utility.

The compositions of the invention can be presented in the form of unit dosage forms containing a defined concentration of soluble fibre. Such unit dosage forms can be selected so as to achieve a desired level of biological activity.

The amount of a soluble fibre required by a subject is determined by biological activity and bioavailability which in turn depends on the formulation, mode of administration, the physicochemical properties of the soluble fibres and whether the soluble fibre is being used as a monotherapy or in a combined therapy. Generally, a daily dose for a human adult should be between 0.1 g and 100 g of freeze-dried or spray-dried powder (however formulated), more preferably the daily dose is between 1 g and 30 g (e.g. about 5 g, 10 g, or 15 g as required).

A solid or semisolid dosage form of the present invention can contain up to about 1000 mg of dried extract containing soluble fibre, for example up to about 800 mg.

The results reported in the Examples elsewhere in this specification illustrate that increasing the amount of soluble fibre in the compositions of the invention increases the effectiveness of the compositions in inhibiting C. difficile adhesion, and thus treating AAD. The results reported in the Examples clearly illustrate that a highly significant reduction in C. difficile adhesion can be achieved using a concentration of soluble fibres of around 50 mg/mL. That said, since higher concentrations of soluble fibres have higher inhibitory activity, it may be preferred to make use of concentrations capable of substantially preventing C. difficile adhesion. Merely by way of example, the results reported herein suggest that C. difficile adhesion may be substantially prevented by using a soluble fibre concentration of around 150 mg/mL.

Additionally or alternatively the ability of compositions of the invention to inhibit C. difficile adhesion, and thus treat AAD, may be increased by the use of specific fractions of the soluble fibre extract described in Example 2.

The frequency of administration will also be influenced by the above mentioned factors and particularly the half-life and of the soluble fibres within the subject being treated. For instance, the half-life will be influenced by the health status of the subject, gut motility and other factors.

The soluble fibre is particularly useful when included in pharmaceutical formulation such as a tablet or a capsule. Such formulations may be required to be enterally-coated if bioavailability dictates this. Known procedures, such as those conventionally employed by the pharmaceutical industry (e.g. in vivo experimentation, clinical trials etc), may be used to establish specific formulations of pharmaceutical compositions and precise therapeutic regimes (such as daily doses of the compounds and the frequency of administration).

It will be appreciated that conventional “nutraceutical” procedures may be employed to create liquid drinks, powder mixes and food-stuffs comprising the soluble fibre.

Daily doses may be given as a single administration (e.g. a daily tablet for oral consumption or as a single liquid drink). Alternatively the soluble fibre used may require administration twice or more times during a day. As an example, a 100 ml orange drink containing 0.1-20 g of spray dried soluble fibre (preferably 0.3-10 g of spray dried soluble fibre and more preferably 0.5-3.0 g) may be used to quench thirst at regular intervals throughout the day and thereby deliver a recommended dose.

It will be appreciated that nutritional products supplemented with soluble fibre represent an ideal means for providing subjects with, or at risk of developing, AAD with soluble fibre according to the present invention. Therefore, according to a fourth aspect of the present invention there is provided a nutritional product for use in the prevention or treatment of AAD comprising a therapeutically effective amount of a soluble fibre derivable from fruit of the Musa spp.

The nutritional product may comprise:
(a) a clear, low viscosity, water-like, stable, ready-to-use, bottled, carbonated or non-carbonated drink; or a concentrated clear liquid for reconstitution containing a non-gelling soluble fibre derivable from fruit of the Musa spp;
(b) a powder/granular mix to be reconstituted with water or any other orally ingestible liquid as a drinkable liquid, containing a non-gelling soluble fibre derivable from fruit of the Musa spp;
(c) a powder/granular mix mixed into a food stuff (e.g. a chocolate bar, lozenge or the like);
(d) a yoghurt-based mixture containing probiotic bacteria and non-gelling soluble fibre derivable from fruit of the Musa spp.; or
(e) a sterile solution in pre-packaged units suitable for enteral feeding

The nutritional product may be as described above and may or may not contain water soluble vitamins, additional mineral supplements, nutritional compounds, antioxidants or flavourings.

The compositions according to the invention are particularly useful for administration to human subjects in hospitals. Hospital clinicians may administer the compositions to a subject before antibiotic therapy is initiated and may continue to administer the composition during the course of antibiotic treatment. Furthermore treatment with the composition may be advisable for a period after the course of treatment has been completed. A clinician will be able to assess what regimen would be most suitable when the health status (age, strength, immuno-status etc) of a subject being treated.

It will be appreciated that the composition may also be recommended by General practitioners/physicians who practice out side hospitals. Pharmacists may also recommend use of the composition when dispensing a course of antibiotics.

It is preferred that subjects treated according to the invention are humans. However it will be appreciated that the compositions will be useful in animals of veterinary importance that have been put on antibiotic treatment. Such animals may include livestock bred for food and also pet animals.

The present invention will be further illustrated, by way of examples, with reference to the accompanying drawings in which:

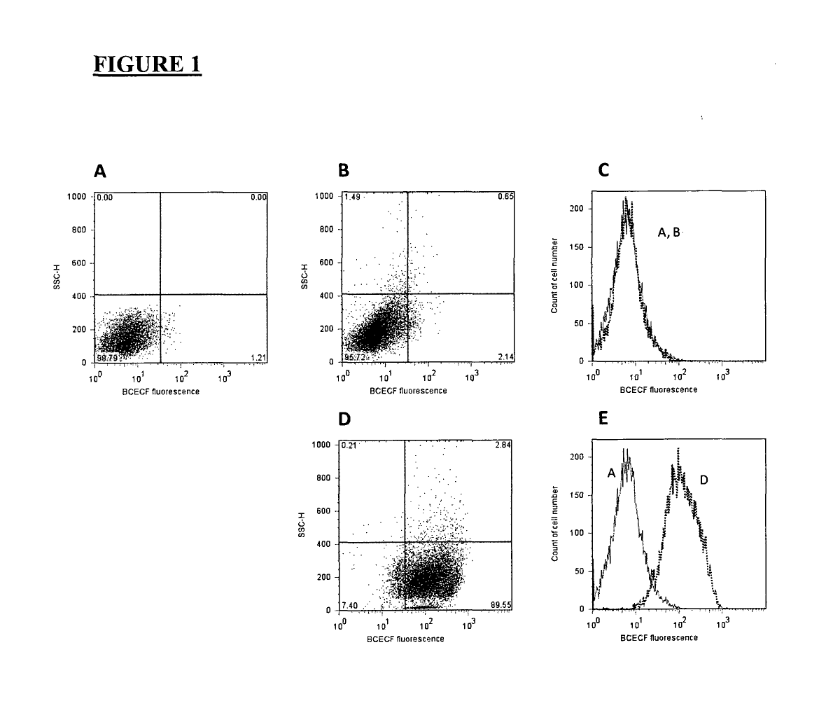

FIG. 1 shows the adherence of C. difficile to Caco-2 cells as discussed in Example 1, illustrated by dotplots of particle side-scatter vs fluourescence (A, B, D) and the accompanying histograms (C, E).

Dotplot A: Caco-2 cells alone
Dotplot B: Caco-2 cells incubated with unstained C. difficile bacteria
Dotplot D: Caco-2 cells incubated with BCECF-stained C. difficile bacteria
Histogram C: Overlay of histograms constructed from the data shown in dotplots A and B
Histogram E: Overlay of histograms constructed from the data shown in dotplots A and D

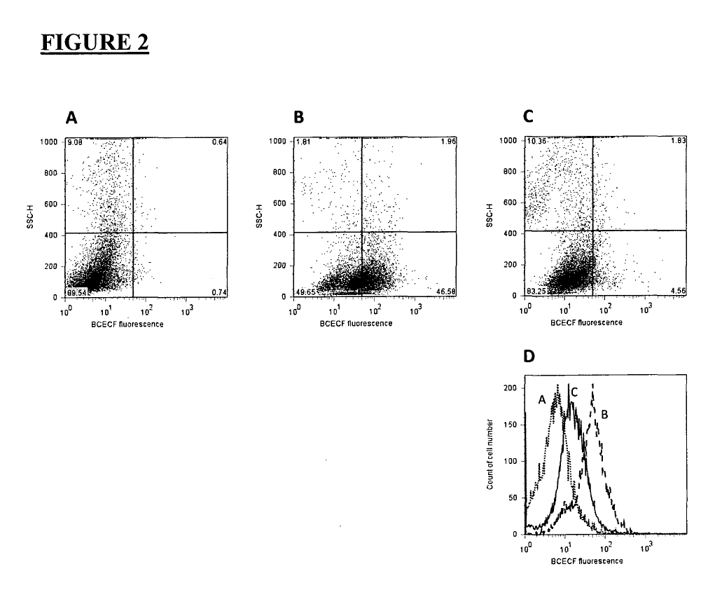

FIG. 2 shows the inhibition of C. difficile adherence to Caco-2 cells by soluble plantain fibre as discussed in Example 1, illustrated by dotplots of particle side-scatter vs fluorescence (A, B, C) and the accompanying histogram D.

Dotplot A: Caco-2 cells alone
Dotplot B: Caco-2 cells incubated with BCECF-stained C. difficile bacteria
Dotplot C: Caco-2 cells incubated with BCECF-stained C. difficile bacteria and soluble plantain NSP
Histogram D: Overlay of histograms constructed from the data shown in dotplots A, B and C.

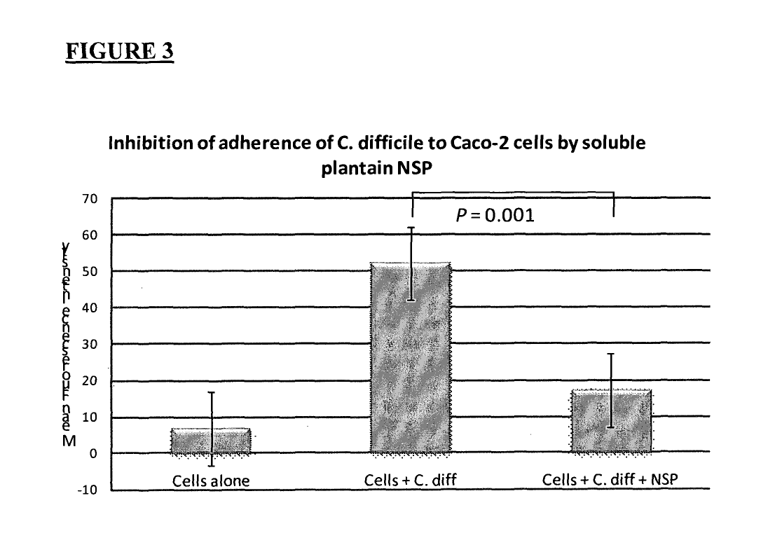

FIG. 3 is a bar chart summarising mean fluorescence intensity data from 4 experiments examining the adherence of C. difficile to Caco-2 cells and inhibition of adherence by soluble plantain fibre as discussed in Example 1.

FIG. 4 is a flow chart of a production process for a preferred composition according to the invention as discussed in Example 2.

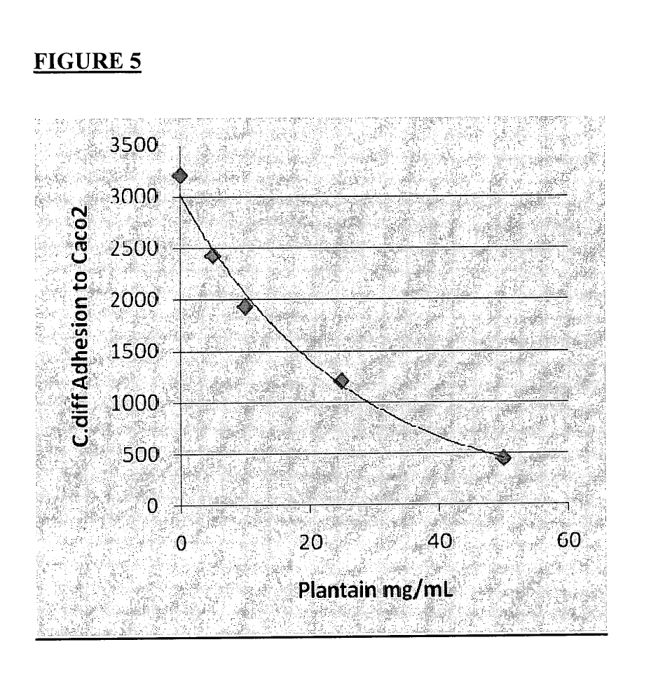

FIG. 5 is a graph plotting C. difficile adhesion to human Caco2 cells (y-axis) against increasing concentrations of soluble fibre (x-axis).

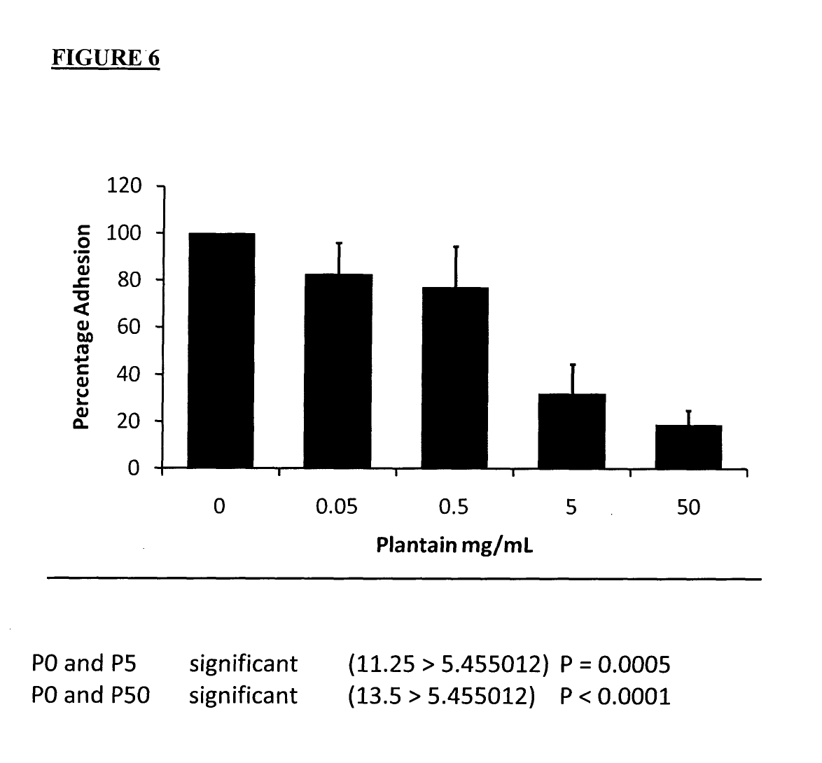

FIG. 6 shows a bar chart comparing percentage adhesion of C. difficile to human Caco2 cells in control cultures and cultures incorporating various concentrations of soluble fibres, and also shows data illustrating the statistical significance of the inhibition of adhesion that may be achieved.

### EXAMPLE 1

The inventors conducted studies with the purpose of investigating the efficacy of compositions according to the invention for inhibiting the adhesion of C. difficile to human ileocaecal epithelial cells.

### 1.1 Methods

### 1.1.1 Cell Culture

Caco-2 human ileocaecal epithelial cells were maintained in DMEM containing glucose supplemented with fetal calf serum (5%), amphotericin B, penicillin/Streptomycin, HEPES buffer and non-essential amino acids. Cells were maintained in polystyrene tissue-culture flasks until 80% confluent. Twenty four hours before adherence assays the medium was removed, cells were washed ×3 in PBS, and cells were incubated for 24 h with antibiotic-free medium. Cells were then washed ×3 with PBS and removed from tissue-culture flasks with an EDTA-trypsin suspension, counted in a haemocytometer and adjusted to the relevant concentration (1×10⁵ cells/ml)

### 1.1.2 Preparation of NSP

Plantain derived NSP (prepared according to the methods described in Example 2) was suspended in sterile PBS to give a concentration of 20 mg/ml (for a final experimental concentration of 10 mg/ml)

### 1.1.3 Bacterial Growth Conditions

C. difficile Strain: O27 (strain number 080042, obtained from Dr Godfrey Smith, Medical Microbiology, University of Liverpool), toxin positive strain was used. C. difficile had been cultured anaerobically on Braziers medium and subsequently stored on Protect beads at −80° C. C. difficile was cultured by placing one protect bead into 50 ml Fastidious Anaerobe Broth (Lab M) and culturing anaerobically without shaking for 36 h at 37° C.

### 1.1.4 Harvesting Bacteria

Bacteria were harvested by centrifugation at 9000 rpm for 5 min and resuspended in sterile PBS. Bacteria in suspension were then quantified by OD reading at 550 nm, and were adjusted to 1×10⁸ bacteria/ml (OD=0.82) for fluorochrome labelling.

### 1.1.5 Fluorochrome

1 mM BCECF/AM stock solution had been divided into 50 μl aliquots which were stored in a well-sealed container and protected from the light with aluminium foil and stored in the −20° C. freezer. For a 1 μM working concentration 10 μl stock solution of BCECF/AM was added for every 10 ml bacterial suspension (1×10⁸ bugs/ml).

Bacteria were labelled with BCECF/AM fluorochrome at 37° C. for 60 min ensuring that tubes were protected from the light with aluminium foil prior to incubation. Excess fluorochrome was removed by 5 sequential washes in PBS followed by centrifugation at 9000 rpm for 5 min. After the penultimate wash, each bacterial suspension was split into 2 falcon tubes before centrifugation. After the final wash bacteria were re-suspended in PBS containing 20 mg/ml plantain NSP, or PBS alone and were incubated for 30 min ensuring tubes were protected from the light using aluminium foil.

### 1.1.6 Adherence Assay

An equivalent volume of 1×10⁵ cells/ml Caco-2 cells were added to the fluorescently labelled bacteria and incubated at 37° C. for 1 h. After incubation, cells were washed by centrifugation at 1200 rpm for 20 min (×3) in PBS to remove any non-adherent bacteria. After the final wash the pellets were resuspended in 1 ml PBS and were transferred into individual eppendorf tubes.

### 1.1.7 Flow Cytometry

Caco-2 cells with and without adherent C. difficile in the presence or absence of soluble plantain fibre were measured in a FACScan flow cytometer (Becton Dickinson, Oxford). A total of 10,000 events was acquired and the data were analysed with the Cell Quest software programme from Becton Dickinson. Mean Fluorescent Intensity (MFI) using the geometric mean of each sample was used to assess bacterial adherence to Caco-2 cells. Students-t test was used to compare differences in bacterial adherence in the presence or absence of soluble plantain fibre.

### 1.2 Results

The abovementioned methods were designed to assess the effect of compositions according to the invention on adherence of labelled C. difficile to human ileocaecal epithelial cells by FACs analysis. A skilled person will appreciate that this represents a good model of adhesion in vivo and would understand that a composition that reduces adhesion represents an agent that would be suitable for treating AAD (a condition characterised by inappropriate adhesion of C. difficile difficile to ileocaecal epithelial cells).

FIG. 1 illustrates the results of control experiments obtained when assessing adherence of C. difficile to Caco-2 cells. All data presented is based on 10,000 cell counts, and no gating was carried out. FIG. 1A shows the profile of Caco-2 cells alone, when the scatter characteristics of the cells is graphed against their fluorescence intensity. A distinct population (>98% of all events) can be observed in the lower left quartile. FIG. 1B shows the profile of Caco-2 cells preincubated with unstained C. difficile bacteria, and demonstrates that almost all the bacteria associate with the Caco-2 cells (no second population can be distinguished) in the low-fluorescence area of the graph. More than 95% of the total events are in the lower left quartile. The full overlap of the two histograms A and B (A=dotted line, B=dashed line) in FIG. 1C further demonstrates that bacteria and Caco-2 cells are occurring as one population. FIG. 1D shows the profile of Caco-2 cells preincubated with stained C. difficile bacteria. In this case, 90% of events counted occur in the lower-right quartile, with 7% remaining in the lower-left quartile. This shows that Caco-2 cells alone, without bacterial attachment and with low fluorescence intensity, could account for 7% of the events counted by the flow cytometer; however the majority (90%) of Caco-2 cells are found associated with the high-fluorescence stained bacteria. The histograms shown in FIG. 1E further illustrate this point.

Thus control experiments show that pre-incubation of Caco-2 cells with C. difficile bacteria leads to association and binding of the bacteria to the cells.

FIG. 2 illustrates the effect of pre-incubation of the cells with a composition according to the invention (plantain derived non starch polysaccharides (NSP)) on subsequent binding of BCECF-stained C. difficile to the Caco-2 cells. FIG. 2A shows the profile of Caco-2 cells alone, occurring in a single population in the lower left quartile. FIG. 2B shows the effect of preincubation with BCECF-stained C. difficile bacteria; as in FIG. 1, a population of associated bacteria and cells can be distinguished in the high-fluorescing, lower right quartile, comprising in this case 47% of all events (binding in this experiment was evidently lower than in the experiment described above, as a larger percentage of events remains in the lower left quartile (low-fluorescence), perhaps due to slight variabilities in the incubation conditions). FIG. 2C shows the effect of preincubating the Caco-2 cells with NSP, before the addition of bacteria. Under these conditions, only a low-fluorescence population is observed (>83% of events in the lower left quartile), indicating that in the presence of NSP, the high-fluorescing bacteria had been washed off the cells before the particles were counted in the flow cytometer, and were not found bound to the cells. FIG. 2D shows the data in histogram format.

This demonstrates that the NSP had the effect of preventing adhesion of C. difficile to Caco-2 cells.

FIG. 3 illustrates average results (n=4) for experiments assessing the adherence of C. difficile to Caco-2 cells and inhibition of adherence by NSP.

This data lead the inventors to the surprising realisation that compositions according to the present invention will have a significant displacing effect on pathogenic bacterium from the gut wall. This has the effect of reducing colonisation by the pathogen and will prevent the development AAD and will also be useful for treating existing AAD.

### EXAMPLE 2

Having established that compositions according to the invention had efficacy for reducing adhesion of C. difficile to epithelial cells of the gut. The inventors went on to develop compositions suitable for use as an investigative medicinal product (suitable for clinical trials) and/or use on commercial basis. The compositions and processes discussed below represent further aspects of the invention.

A standardised mixture of naturally-occurring polysaccharides derived from plantain fruit (Musa sapientum), was formulated with maltodextrins and natural flavourings to produce a free-flowing powder which can be dissolved in water for oral administration. The polysaccharides are water-extractable from fresh or dried plantain fruit tissue, and are not related to starch in structure. They are thus known as soluble non-starch polysaccharides (NSP). These polysaccharides are cell wall derived. The cell walls of edible plantain fruits are mainly primary walls in character (excepting fibrous elements associated with skin) and conform to the typical description of type I cell walls. That is, matrix polysaccharides are largely of a pectic nature (acidic), with smaller quantities of varied neutral polysaccharides also present. In soluble plantain NSP, the acidic polysaccharides present comprise a group of pectic polysaccharides with associated arabinans and xylans. The neutral polysaccharides include arabinoxylans, mannans, glucomannans and xyloglucans.

### 2.1 Manufacture of a Composition According to the Invention

A preferred free-flowing powder composition according to the present invention was obtained after aqueous extraction of green plantain flour. The process involves six stages: a detailed flowchart is shown in FIG. 4.

### Stage 1: Reconstitution.

The dry plantain flour is weighed into the vessel compartment of a Limitech high-shear mixer and mixed with reverse-osmosis purified water in a ratio of 1:2 until a homogenous suspension is formed.

### Stage 2: Starch Swelling and Gelatinisation

The homogenised mixture is heated to between 90 and 100° C. (steam-heated vessel jacket) and held at temperature for 10 minutes with continuous high-shear mixing. The starch present in the plantain flour swells and gelatinises with heating, and forms a very viscous elastic gel. Continuous high-shear mixing breaks the gel and prevents the mixture from sticking to the sides of the vessel during the heating step. After 10 minutes, the vessel is cooled by means of cooling water to 25° C.

### Stage 3: Starch Liquefaction

Fungal α-amylase (Fungamyl, Novozymes Corporation) is used to liquefy the starch gel. This source of α-amylase is preferred because it is a low-temperature enzyme with an optimum operating temperature of 25-28° C., at pH 6-7. High temperatures for extended periods result in polysaccharide degradation and are avoided; thus the low temperature enzyme is ideal. On addition of enzyme, the starch gel is immediately and irreversibly broken, as the starch molecules are degraded to smaller molecular weight maltodextrins. The liquefaction process is complete within 2 hours at 25-28° C., the optimal temperature range for fungal α-amylase. After 2 hours, the liquefied mixture is diluted to 1000 L with RO water, and heated to 72° C. for 20 minutes to fully inactive the Fungamyl enzyme and to pasteurise the mixture. The mixture is then chilled quickly to 25-30° C.

### Stage 4: Separation of Soluble and Insoluble NSP

The mixture is pumped into a holding tank and diluted further to 3000 L with RO water so that the final mixture contains 4.3-4.5% solids (measured using a Labwave microwave-based system). The mixture is then separated on a centrifugal separator (KNA3, Alfa Lavaal) in two steps, so that an overall split of supernatant:sludge of 80%:20% (w/w) is achieved. The supernatant contains 3.4-3.5% total solids, but <0.1% insoluble solids. The sludge contains 4.7-4.8% total solids, of which >50% are insoluble solids.

### Stage 5: Removal of Low-Molecular Weight Starch Degradation Products

A small amount of free glucose and maltose may be produced during the Fungamyl enzyme digestion. These, as well as other low molecular weight compounds such as sucrose, free amino acids, are removed from the soluble NSP by nanofiltration using spiral-wound membranes. The membranes permit small molecules (MW<300 Da) to pass through, while retaining all other material inside the membrane. Thus water is removed from the mixture concomitantly, and unless replaced (a process called ‘diafiltration’), concentration of the mixture will occur.

Nanofiltration is carried out without diafiltration until the % solids of the retained material has risen from 3.4-3.5% to 6.0%. During this period, the theoretical amount of permeable material is calculated from the % solids content and flow rate of the permeate stream. Once the retentate has reached 6.0% solids, diafiltration is commenced, adding water back into the retentate at the same rate as it is being removed. This continues until 90% of the theoretically permeable material has been removed, at which point the process is halted. The retentate is recovered, and the permeate is discarded.

### Stage 6: Drying

The retentate fraction, containing soluble NSP and large MW maltodextrins, is concentrated by evaporation under reduced pressure at a temperature of 40 C until it reaches 30% solids. It is then passed to a spray-drier, incorporating an on-line pasteurisation step before it reaches the drier. The mixture is spray-dried without agglomeration to produce a fine dry powder with a particle size distribution of 50-100μ and a bulk density of 175 g/L. The powder is automatically bagged into foil-backed packaging in 20 kg portions, and each portion is closed by heat-sealing.

### 2.2 Characteristics of the Composition Manufactured According to the Methods Described in 2.1

A water-soluble extract from the edible fruits of the plantain (Musa spp. AAB group (Horn)) was formed comprising a mixture of non-starch polysaccharides and low dextrose-equivalence maltodextrins.

The non-starch polysaccharides present are cell wall derived, and occur naturally in both fresh plantains and processed plantain products (e.g. plantain flour, plantain chips). The mixture contains pectic polysaccharides, arabinans, arabinoxylans, xyloglucans, mannose-containing polysaccharides such as galacturonomannans and glucuronomannans, and a range of heterogenous neutral polysaccharides.

The maltodextrin component is derived from the starch component of the plantain source material, which has been liquefied and cleaved into lower molecular weight structures by the action of α-amylase. This maltodextrin component is retained in the composition due to its usefulness as a drying aid.

The composition is substantially free of low molecular weight sugars such as glucose and fructose; the molecular size distribution ranges approximately from 800-5000 kDa.

The composition is in a dry powder format, with moisture content <4% and an off-white colour. It is soluble in water (1 g/20 g). In aqueous solution at 1 g/20 g, it is characterised by a pH of ˜6.8, a slightly turbid appearance of indeterminate colour, and a neutral smell and taste. In solution it is stable to flash pasteurisation and UHT treatments; long heat treatments (e.g. 1 hour at 90 C) lead to cleavage of some heat-labile polysaccharide bonds. Further physical characteristics are summarised in Table 3.

TABLE 3
General physical characteristics

| | |
|---|---|
| Dry matter (%) | 96 |
| Moisture (%) | 4 |
| Bulk density (g/L) | 175 |
| Particle size (μ) | 50-100 |
| Solubility (g/g H₂O) | 1/20 |
| pH (5% solution) | 6.6-6.8 |
| Colour | White/cream |

Table 4 provides more detail on the chemical composition of the powder manufactured according to the processes described in 2.1.

TABLE 4

| | |
|---|---|
| Source material | Plantain flour (Ecuador) |

Added ingredients

| | |
|---|---|
| Fungamyl (Novozymes) | Food grade starch-degrading |
| | enzyme (-amylase), fully |
| | deactivated |

Nutritional values

| | |
|---|---|
| Protein | 0.6 g/100 g |
| Total carbohydrate | 82.3 g/100 g |
| of which sugars (maltodextrin, | 25.4 g/100 g |
| expressed as glucose) | |
| of which starch | <0.1 g/100 g |
| of which complex polysaccharides | 56.9 g/100 g |
| Dietary fibre (AOAC) | 1.9 g/100 g |
| Fat | <0.1 g/100 g |
| Ash | 11.7 g/100 g |
| Moisture | 5.4 g/100 g |
| Energy | 1377 kJ/100 g or 324 kcal/100 g |
| Calcium | 0.24 g/100 g |
| Potassium | 0.98 g/100 g |
| Magnesium | 0.11 g/100 g |
| Sodium | 3.3 g/100 g |

Physical properties

| | |
|---|---|
| Colour | White/cream |
| Taste | Slightly sweet, no strong taste |
| Texture | Fine particles, flour-like |
| | consistency |
| Solubility | Water soluble, alcohol insoluble |
| Bulk density | 175 g/L |
| Particle size | Not yet determined |

Microbiological properties

| | |
|---|---|
| Total Viable Count cfu/ml | <1000 |
| Salmonella/Listeria | Absent (<1/25 g) |
| Staphylococcus aureus | Absent (<1/10 g) |
| Enterobacteria | <10/10 g |
| Yeasts/moulds | <100/10 g |
| Storage conditions | Store in dry conditions at room |
| | temperature. Hygroscopic. |
| Use | For use as a food ingredient or |
| | medical substance at a specified |
| | level of 5 g per unit dose |

### EXAMPLE 3

A protocol was developed to test the ability of a composition according to the invention used in combination with a probiotic (a preferred combination referred to above). The protocol was adapted from Hickson, M. et al. 2007. “Use of probiotic Lactobacillus preparation to prevent diarrhoea associated with antibiotics: randomised double blind placebo controlled trial”. BMJ; 335:80

A formulation such as that described in Example 2 could be tested in a randomised double blind, placebo controlled trial following the protocol described in the following Example.

### 3.1 Methods

The protocol was set up to examine whether or not a plantain NSP preparation (e.g. as prepared in Example 2) would reduce the incidence of antibiotic associated diarrhoea and C. difficile associated diarrhoea.

The primary outcome measured according to the protocol was the occurrence of diarrhoea, recorded by nursing staff and authenticated by researchers, where diarrhoea is defined as more than two liquid stools a day for three or more days in quantities in excess of normal for each patient.

The secondary outcome measured according to the protocol was the occurrence of C. difficile infection, defined as an episode of diarrhoea combined with the detection of toxins A or B, or both, from a stool sample (enzyme immunoassay kit, Meridian Bioscience, Ohio, USA).

### 3.1.1 Participants

Recruitment of patients to be carried out from Liverpool hospitals: The Royal Liverpool Hospital and Aintree NHS Trust. Patients should be recruited mainly from orthopaedic, medical, and care of the elderly wards, and include inpatients aged over 50 who are prescribed antibiotics (single or multiple antibiotics, oral or intravenous) and are able to take food and drink orally. Patients must be able to give written informed consent.

### 3.1.2 Exclusion Criteria

Diarrhoea on admission or within the preceding week, reported recurrent diarrhoea, or bowel pathology that could result in diarrhoea; intake of high risk antibiotics (clindamycin, cephalosporins, aminopenicillins) or more than two courses of other antibiotics in the past four weeks to exclude pre-existing diarrhoea associated with antibiotic use; severe life threatening illness, immunosuppression, bowel surgery; artificial heart valve, history of rheumatic heart disease, or history of infective endocarditis; regular probiotic treatment before admission; and lactose intolerance or intolerance to dairy products.

### 3.1.3 Interventions

Treatment group: a 200 ml drink comprising 5 g NSP formulation as described in Example 2, dissolved in water.

Placebo group: a 200 ml drink comprising 5 g sucralose, dissolved in water.

Drinks should be taken twice per day, either half an hour before or one to two hours after meals. Participants should begin using the drinks within 48 hours of starting antibiotic therapy and continue doing so for one week after they stop taking antibiotics. Researchers will verify participants' consumption and record missed or refused drinks to assess compliance.

### 3.1.4 Study Plan

The admitting medical team should identify potential patients who have been prescribed antibiotics and the researchers approach them within 48 hours of the first antibiotic dose. Once informed consent is obtained, baseline data is to be collected and the randomised study drink prescribed. The hospital pharmacy will dispense the drink. A baseline stool sample will be collected to screen for asymptomatic C. difficile carriage. Bowel movements will be monitored with stool charts, checked every weekday for accuracy. When there is evidence of diarrhoea a stool sample will be analysed for C. difficile toxin.

Once the antibiotic course is finished a final week of study drink will be dispensed and a final follow-up date fixed for four weeks later. Patients who were discharged taking antibiotics will be provided with enough drink on discharge to cover the period they have to take antibiotics plus one week. Researchers will follow up participants for four weeks from discharge with weekly phone calls to ask about diarrhoea and compliance. If participants have diarrhoea, the researchers will collect a further stool sample to check for C. difficile toxin.

### 3.1.5 Sample Size

With α=0.05 and a power of 90% to detect an absolute difference of 20% between the proportion of patients with antibiotic associated diarrhoea in the placebo (assumed at 30%) and probiotic (assumed at 10%) groups, it is estimated that a sample size of 164 is needed (82 in each group).

### 3.1.6 Randomisation

An independent statistician will generate the random allocation sequence, stratified for hospital, sex, and two age groups (50-69 and ≧70). The sequence will be given to the pharmacy on each site.

### 3.1.7 Blinding

The study treatments will be supplied identified only by study labels to identify the patient, the drink's “use by” date, and storage instructions. Patients and researchers will be blind to the study drink identity.

Potential bias through unblinding is possible but unlikely, and the outcome measure should be checked and agreed between two or more people. Microbiology staff grouping assessed occurrence of C. difficile by analysis of a stool sample from patients who had diarrhoea will be blind to the study.

### 3.1.8 Statistical Methods

Fisher's exact test should be used to compare rates of antibiotic associated diarrhoea and C. difficile associated diarrhoea, and logistic regression (block entry with removal of non-significant variables) to establish which factors influenced the occurrence of diarrhoea and to estimate the adjusted odds ratio for treatment effect.

### EXAMPLE 4

### Production of a Powder Mix of Plantain Soluble Fibre According to the Invention for Resale

3.0 g of freeze dried soluble fibre powder (Example 2) was mixed with 0.5 g powdered citric acid, 26.3 g of granulated sugar and 0.2 g of a standard spray-dried mix of flavouring.

This mixture represents a free-flowing powder formulation (containing 3.0 g of soluble fibre) that is suitable for packaging in a sachet. The powder mix may be diluted to taste and drunk when required by a subject suffering from IBD.

### EXAMPLE 5

### Production of an Orange Drink for Use According to the Invention

(a) 100 ml of crude preparation (prepared according to method 1.1.12.2) was mixed with 100 mls of double concentrate orange juice (orange juice concentrate diluted in water to double strength).
(b) 3.5 g of freeze-dried powder (prepared according to Example 2) was dissolved in 100 mls of orange juice (or alternatively with orange juice concentrate and water).

The orange drink preparations (a or b) may be consumed by a subject immediately, refrigerated for later consumption or sealed in a bottle or carton for a longer shelf life. It will be appreciated that orange juice may be readily substituted with a palatable alternative.

### EXAMPLE 6

### Production of a Nutritional Liquid Formulation Containing Plantain Soluble Fibre for Use in Enteral Feeding

A liquid nutritional mixture may be produced for use in enteral feeding in which the total content of the NSP (prepared according to Example 2) is between 0.1 and 10% of the total dry matter content of the feed mixture. The NSP may be mixed with saline or an aqueous solution, and other vitamins, minerals and nutrients may be included as required for enteral feeding of subjects. Such enteral liquid formulations may be packaged in pouches (e.g containing 100 mls-2,000 mls) for use in a drip or for insertion into a pump feed.

### EXAMPLE 7

### Production of a Liquid Formulation Containing Plantain Soluble Fibre and Probiotic Bacteria for Use According to the Invention

A mixture containing both NSP and probiotic bacteria may be produced in which the total content of the NSP (method as in Example 2) is between 5% and 15% of the final mixture (w/v), and the mixture also contains probiotic bacteria selected from the species Bifidobacteria, Lactobaccilli and Streptococc in the concentration 1×10⁸ colony forming units/ml. The NSP may be mixed with a preformulation yoghurt drink, containing appropriate flavours, sweeteners and acidity regulators, and the mixture thus formed pasteurised at 121° C. for 3 minutes. The prebiotic bacteria may then be post-dosed into this mixture at the concentration indicated and the final mixture packaged into single-serve portions and heat-sealed for use within 21 days.

### EXAMPLE 8

The inventors undertook further studies to illustrate the effectiveness of compositions of the invention in inhibiting adhesion of C. difficile to human cells, and thus treating AAD. These studies used a highly sensitive bacterial culture assay which assesses adhesion of live bacteria to the surface of human cells.

### 8.1 Methods

### 8.1.1 Cell Culture

Caco-2 human ileocaecal epithelial cells were maintained in DMEM supplemented with L-glutamine, 10% fetal calf serum, amphotericin B, and penicillin/Streptomycin, Cells were seeded into 24 well tissue culture plates at 4×10⁵.cells per well, and maintained in DMEM (plus FCS and antibiotics) until a confluent monolayer was established. Cells were then washed twice with PBS prior to exposure to bacteria.

### 8.1.2 Preparation of NSP

Plantain derived NSP (prepared according to the methods described in Example 2) was suspended in sterile PBS to give experimental solutions having the concentrations set out below (the experimental solutions being representative of compositions of the invention):
0.05 mg/mL
0.5 mg/mL
10 mg/mL
25 mg/mL
50 mg/mL

PBS alone was used as a control (noted as 0 mg/mL in the accompanying Figures).

### 8.1.3 Bacterial Growth Conditions

C. difficile Strain: O27 (strain number 080042, obtained from Dr Godfrey Smith, Medical Microbiology, University of Liverpool), toxin positive strain was used. C. difficile had been cultured anaerobically on Braziers medium and subsequently stored on Protect beads at −80° C. C. difficile was cultured by placing one protect bead into 50 ml Fastidious Anaerobe Broth (Lab M) and culturing anaerobically without shaking for 36 h at 37° C.

### 8.1.4 Harvesting Bacteria

Bacteria were harvested by centrifugation at 9000 rpm for 5 min and resuspended in sterile PBS. Bacteria in suspension were then quantified by OD reading at 550 nm, and were adjusted to 1×10⁸ bacteria/ml (OD=0.82) for fluorochrome labelling.

### 8.1.5 Fluorochrome Labelling

1 mM BCECF/AM stock solution had been divided into 50 μl aliquots which were stored in a well-sealed container and protected from the light with aluminium foil and stored in the −20° C. freezer. For a 1 μM working concentration 10 μl stock solution of BCECF/AM was added for every 10 ml bacterial suspension (1×10⁸ bugs/ml).

Bacteria were labelled with BCECF/AM fluorochrome at 37° C. for 60 min ensuring that tubes were protected from the light with aluminium foil prior to incubation. Excess fluorochrome was removed by 5 sequential washes in PBS followed by centrifugation at 9000 rpm for 5 min. After the penultimate wash, each bacterial suspension was split into 2 falcon tubes before centrifugation. After the final wash bacteria were re-suspended in the experimental plantain NSP solutions described above, or in PBS alone.

### 8.1.6 Adherence Assay

The Caco2 monolayers were infected with approximately 7×10⁶ labelled bacteria suspended in the experimental NSP solutions or PBS. Monolayers were exposed to bacteria for four hours to allow adherence to occur. At the end of this period the monolayers were washed with PBS to remove non-adherent bacteria from the cultures. The Caco2 cells and bacteria were then incubated in sterile saline for one hour prior to cell lysis.

The cell monolayers were lysed by the addition of deionized water containing 1% Triton X-100 for five minutes. The adherent bacteria present in each sample were then enumerated by overnight culture of the lysates produced on LB agar maintained at 37° C., followed by a count of the number of colony-forming units present. The results of this study are shown in FIGS. 5 and 6.

### 8.2 Results

Each of the experimental solutions (representative of compositions of the invention) tested exhibited an ability to inhibit C. difficile adhesion. The inhibitory effectiveness of the solutions increased with increasing soluble fibre content.

FIG. 5 plots C. difficile adhesion to human Caco2 cells (y-axis) against increasing concentrations of soluble fibre (x-axis). It can be seen that each of the experimental solutions tested (5 mg/mL, 10 mg/mL, 25 mg/mL and 50 mg/mL of soluble plantain fibres shown) was able to inhibit C. difficile adhesion as compared to the level of adhesion found in the absence of soluble fibres (PBS controls shown as 0 mg/mL), and that the level of inhibition achieved increased with concentration of the soluble fibre. It may be expected that extrapolation of the curve produce to the point at which it crosses the 0 value of the y-axis will allow identification of the concentrations of soluble fibre able to substantially prevent C. difficile adhesion.

FIG. 6 shows a bar chart comparing percentage adhesion of C. difficile to human Caco2 cells in control cultures and cultures incorporating various concentrations of soluble fibres. These are calculated as a percentage of the adhesion that occurs in the absence of soluble fibres (PBS controls, shown as 0 mg/mL).

The results set out in FIG. 6 are as follows:

| | Concentration of soluble fibre | | | | |
|---|---|---|---|---|---|
| | | 0.05 | 0.5 | | |
| | 0 mg/mL | mg/mL | mg/mL | 5 mg/mL | 50 mg/mL |
| Average % | 100 | 82.88399 | 77.46639 | 32.40795 | 19.09522 |
| adhesion | | | | | |
| Standard | 0 | 13.19831 | 17.4209 | 12.32411 | 5.934614 |
| error | | | | | |

The results shown in FIGS. 5 and 6 summarise the data obtained in five studies.

Solutions comprising 5 mg/mL soluble fibre and 50 mg/mL soluble fibre both exhibited significant inhibition as compared to PBS controls (p=0.0006 and p<0.0001 respectively). This ability to significantly inhibit C. difficile adhesion indicates that the compositions of the invention may be used to inhibit C. difficile colonisation, and thus prevent or treat AAD. The method described herein also provides a useful tool for the identification of amounts of soluble fibre that will exhibit therapeutic activity.

1. A composition comprising a therapeutically effective amount of a soluble fibre derivable from fruit of the Musa spp for use as a medicament for preventing or treating antibiotic associated diarrhoea (AAD).

2. The composition according to claim 1 wherein the soluble fibre is derivable from an aqueous solution decantable from homogenised fruit.

3. The composition according to claim 1 wherein the soluble fibre is derived from boiling the fruit.

4. The composition according to claim 1 wherein the soluble fibre is treated to remove starch.

5. The composition according to claim 1 wherein the soluble fibre is from fruit of the Musa spp.

6. The composition according to claim 1 wherein the soluble fibre is essentially as defined in Example 2.

7. The composition according to claim 1 Wherein the therapeutically effective amount of soluble fibre is an amount sufficient to inhibit adhesion of C. difficile.

8. A method for the treatment of antibiotic associated diarrhoea (AAD) comprising administering to a subject in need of such treatment a therapeutically effective amount of a soluble fibre derivable from fruit of the Musa spp.

9. A nutritional product for use in the prevention or treatment of antibiotic associated diarrhoea (AAD) comprising a therapeutically effective amount of a soluble fibre derivable from fruit of the Musa spp.

10. A nutritional product according to claim 9 in the form of a beverage or drink.

11. The nutritional product according to claim 10 comprising between 1 g/100 ml and 30 g/100 ml of soluble fibre.

12. A nutritional product according to claim 9 in the form of a powder or powder mix.

13. A nutritional product according to claim 9 in the form of a food bar or other solid food stuff.

14. A nutritional product according to claim 9 in the form of a sterile solution for enteral feeding.

15. A method of preparing a composition, comprising non-starch soluble fibre, from dry flour comprising the steps of:
(1) reconstituting the flour;
(2) Starch swelling and gelatinisation;
(3) Starch liquefaction;
(4) Separation of soluble and insoluble non-starch polysaccharides;
(5) Removal of low-molecular weight starch degradation products; and
(6) Drying.

16. The method according to claim 15 wherein steps (1)-(6) are as defined in Example 2 or FIG. 4.
